(19) United States
(12) Patent Application Publication
(10) Pub. No.: US 2005/0123799 A1
(43) Pub. Date: Jun. 9, 2005

(54) Luminescent material and organic electroluminescent device

(76) Inventor: Yuji Hamada, Ikoma-gun (JP)

Correspondence Address: KUBOVCIK & KUBOVCIK, SUITE 710, 900 17TH STREET NW, WASHINGTON, DC, 20006, US

(21) Appl. No.: 10/952,498

(22) Filed: Sep. 29, 2004

(30) Foreign Application Priority Data
Sep. 30, 2003 (JP) 2003-342603

(51) Int. Cl. H05B 33/14; C09K 11/06; C07D 277/66

(52) U.S. Cl. 428/690; 428/917; 313/504; 313/506; 257/89; 548/156

(57) ABSTRACT

A red-orange luminescent material represented by General Formula (1): 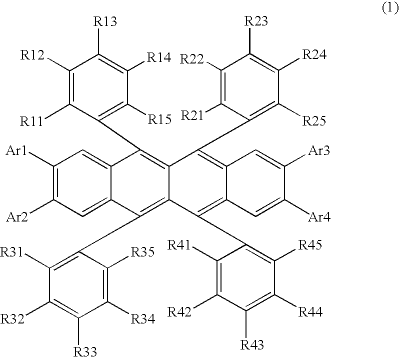 wherein R11 to R15, R21 to R25, R31 to R35, R41 to R45, and Ar1 to Ar4 are as defined in the specification.

### BACKGROUND OF THE INVENTION

1. Field of the Invention

The invention relates to a luminescent material capable of being used as a luminescent material in an organic electroluminescent device (hereinafter referred to as an organic EL device) and relates to an organic EL device using such the material.

2. Description of the Related Art

Organic EL devices generally have a laminated structure in which a carrier-transporting layer (an electron-transporting layer and/or a hole-transporting layer) and a light-emitting layer are provided between a hole-injecting electrode and an electron-injecting electrode.

Such the organic EL devices can produce visible light of from blue to red by selection of organic materials for forming the light-emitting layer. Thus, full-color display can be achieved using organic EL devices that emit monochromic red, green and blue light (the three primary colors of light: RGB (Red-Green-Blue)), respectively.

Among the three colors of light, green and blue of light can be stably produced from organic EL devices. On the other hand, it has conventionally been difficult to produce red-light of high brightness and high efficiency.

It is known that some naphthacene derivatives such as rubrene can emit strong fluorescence and can be used as a luminescent material for a high-brightness organic EL device (Japanese Patent Application Laid-Open (JP-A) No. 2003-55652). However, rubrene emits light of yellow to orange, not red-orange light necessary for full-color display.

Thus, investigations have been made on the naphthacene derivative such as rubrene for the purpose of producing longer wavelength emission. Such investigations include the change of the substituent in the naphthacene derivative and the change of the basic skeleton from naphthacene to pentacene etc. (for example, see the brochure of International Publication No. WO99/57221).

Such methods can produce longer wavelength emission and thus red-orange light. However, such methods have not yet achieved any improvement in emission lifetime of the devices.

### SUMMARY OF THE INVENTION

It is an object of the invention to provide a luminescent material capable of emitting red-orange light and having good life properties and to provide an organic EL device using such the material.

The invention is directed to a luminescent material represented by General Formula (1):

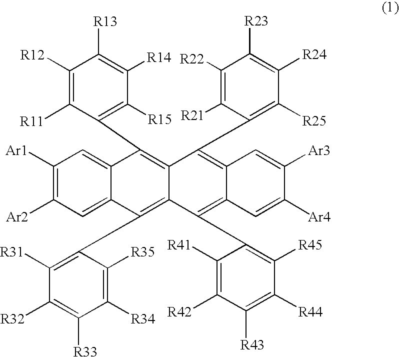

wherein R11 to R15, R21 to R25, R31 to R35, and R41 to R45 are identical to or different from each other and are each hydrogen, an alkyl group represented by —CₙH₂ₙ₊₁ wherein n is an integer of 1 to 10, an alkoxy group represented by —OCₙH₂ₙ₊₁ wherein n is an integer of 1 to 10, a dialkylamino group represented by —N(CₙH₂ₙ₊₁)₂ wherein n is an integer of 1 to 10, fluorine, chlorine, bromine, iodine, a cyano group, or a heterocyclic group represented by Formula (2):

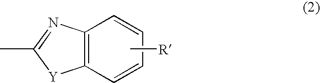

wherein Y is oxygen (O) or sulfur (S); and R′ is hydrogen, an alkyl group represented by —CₙH₂ₙ₊₁ wherein n is an integer of 1 to 10, an alkoxy group represented by —OCₙH₂ₙ₊₁ wherein n is an integer of 1 to 10, a dialkylamino group represented by —N(CₙH₂ₙ₊₁)₂ wherein n is an integer of 1 to 10, fluorine, chlorine, bromine, iodine, a cyano group, or a phenyl group; at least one of R11 to R15, R21 to R25, R31 to R35, and R41 to R45 is the heterocyclic group; and Ar1 to Ar4 are identical to or different from each other and are each an aromatic substituent selected from the following group:

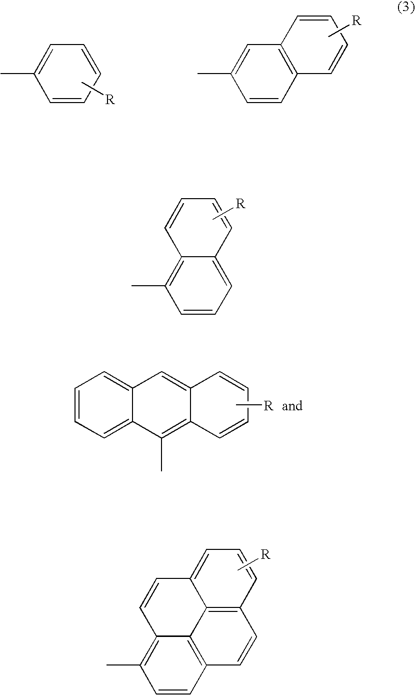

wherein R is hydrogen, an alkyl group represented by —CₙH₂ₙ₊₁ wherein n is an integer of 1 to 10, an alkoxy group represented by —OCₙH₂ₙ₊₁ wherein n is an integer of 1 to 10, a dialkylamino group represented by —N(CₙH₂ₙ₊₁)₂ wherein n is an integer of 1 to 10, fluorine, chlorine, bromine, iodine, a cyano group, or a phenyl group.

The luminescent material of the invention is capable of emitting red-orange light (with a wavelength of 590 to 600 nm) and having good life properties. Examples of such the material include the luminescent materials represented by Formulae (4) to (6) below, specifically.

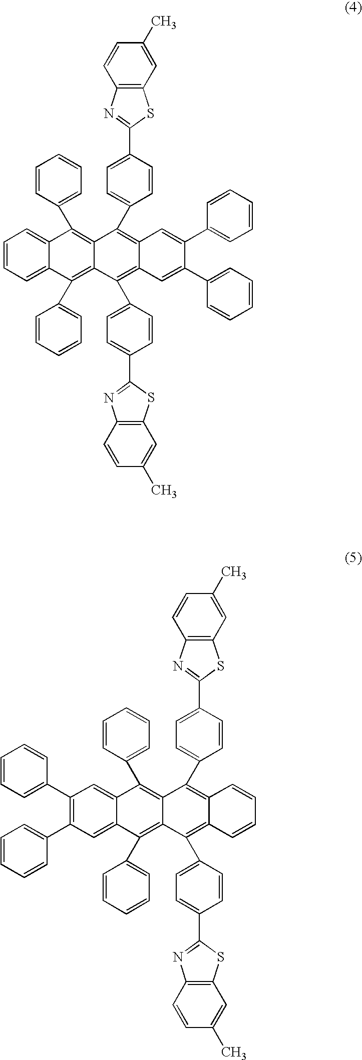

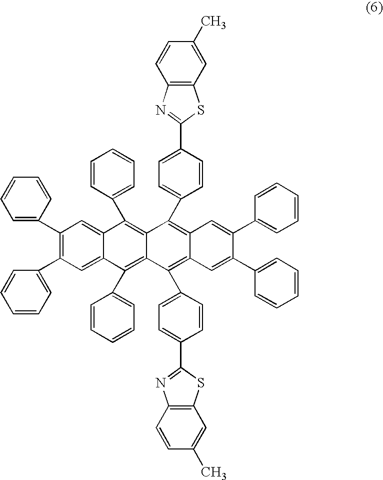

The invention is also directed to an organic EL device comprising a light-emitting layer that contains the luminescent material of the invention. Specifically, the invention is directed to an organic EL device, comprising: a pair of electrodes; and a light-emitting layer sandwiched between the pair of electrodes, wherein the light-emitting layer is a red-orange light emitting layer and contains the luminescent material according to the present invention.

The light-emitting layer containing the luminescent material of the present invention can form an organic EL device capable of emitting red-orange light and having good life properties.

Another aspect of the present invention is directed to an organic EL device, comprising a green light-emitting layer and a blue light-emitting layer in addition to the red-orange light-emitting layer, whereby the device is capable of full-color display.

The luminescent material represented by Formula (4) according to the present invention may be synthesized by reactions represented by the following formulae:

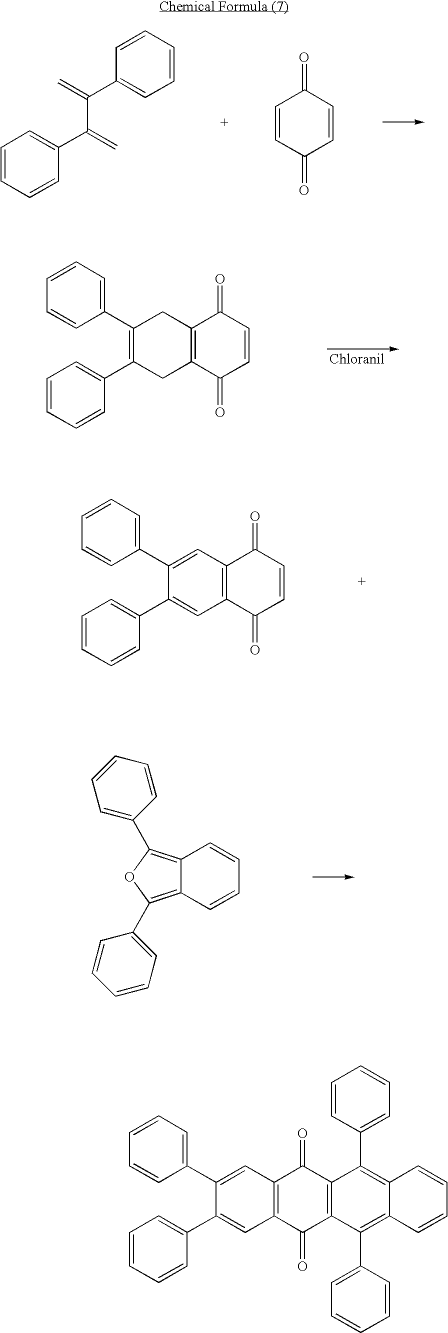

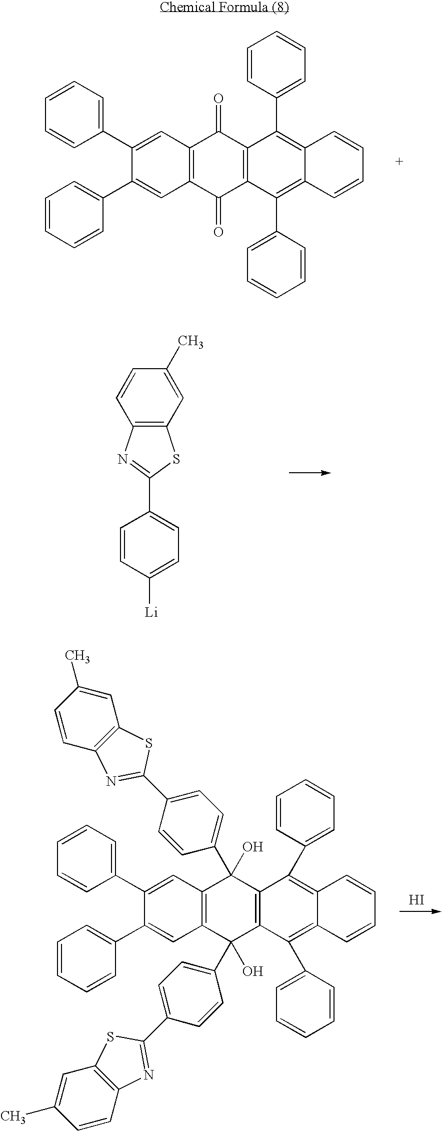

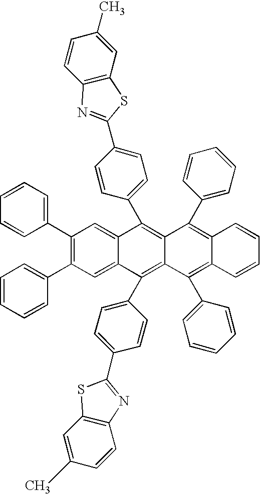

The lithium compound for use in the reaction represented by Chemical Formula (8) may be synthesized by the method represented by the following formula:

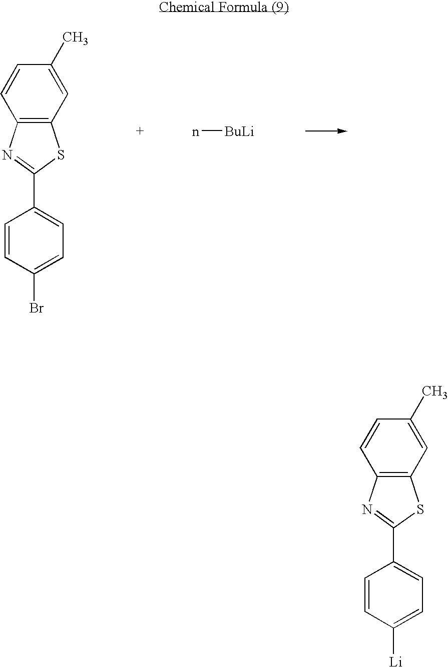

According to the present invention, any other luminescent material may also be produced by a similar method to the above synthesis method.

The luminescent material capable of emitting red-orange light and having good life properties can be obtained according to the present invention.

According to the present invention, the organic EL device has a light-emitting layer capable of emitting red-orange light and having good life properties.

### BRIEF DESCRIPTION OF THE DRAWING

FIG. 1 is a schematic diagram showing an example of the organic EL device according to the present invention.

### DESCRIPTION OF THE PREFERRED EMBODIMENTS

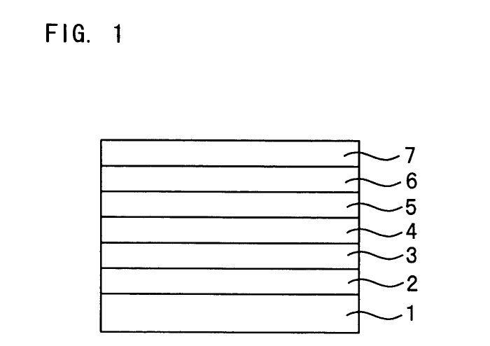

FIG. 1 is a schematic diagram showing an example of the organic EL device according to the present invention. As shown in FIG. 1, a hole-injecting electrode 2 is formed on a substrate 1. For example, a transparent substrate such as a glass substrate is used as the substrate 1. For example, a thin film of an electrically conductive oxide such as indium-tin oxide (ITO) is used as the hole-injecting electrode 2.

A hole-injecting layer 3 and a hole-transporting layer 4 are formed on the hole-injecting electrode 2. A light-emitting layer 5 is formed on the hole-transporting layer 4. The light-emitting layer 5 is generally composed of a host material and a luminescent dopant material.

An electron-transporting layer 6 is formed on the light-emitting layer 5, and an electron-injecting electrode 7 is formed on the electron-transporting layer 6.

The luminescent material of the invention is used in the light-emitting layer 5 to form the organic EL device according to the present invention. The luminescent material of the invention is capable of producing red-orange light emission, and thus the organic EL device is capable of emitting red-orange light.

The light-emitting layer 5 may comprise a red-orange light-emitting layer containing the luminescent material of the invention, a blue light-emitting layer and a green light-emitting layer, which are each formed on the substrate, so as to form an organic EL full-color display device capable of displaying the three colors RGB.

### EXAMPLES 1 to 9

The hole-injecting layer, the hole-transporting layer, the light-emitting layer, the electron-transporting layer, and the cathode (the electron-injecting electrode) were formed using the materials shown in Table 1. In the table, each parenthesized value indicates the thickness (nm) of each layer.

Concerning the luminescent material used in the light-emitting layer, Compounds 1, 2 and 3 are the luminescent materials represented by Formulae (4), (5) and (6), respectively, according to the present invention.

NPB used in each of the hole-transporting layer and the light-emitting layer is N,N′-di(naphthacene-1-yl)-N,N′-diphenylbenzidine, which has the following structural formula:

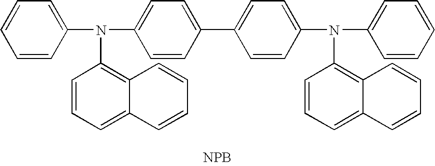

In some cases, the light-emitting layer contains tBuDPN which is 5,12-bis(4-tert-butylphenyl)naphthacene having the following structural formula:

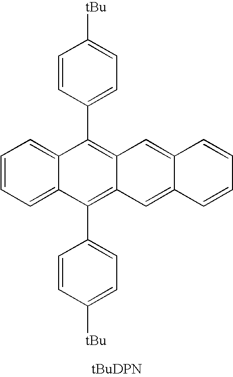

Rubrene used in the light-emitting layer is a compound having the following structural formula:

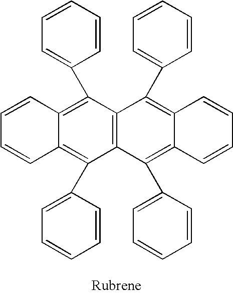

Alq used in the electron-transporting layer is tris(8-hydroxyquinolinato)aluminum having the following structural formula:

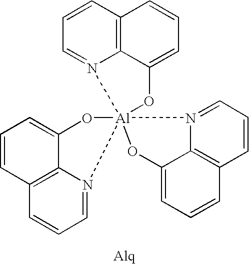

The cathode (the electron-injecting electrode) is formed by laminating LiF and Al.

The light-emitting layer comprises NPB as a host material and Compound 1, 2 or 3 as a luminescent dopant. The each of tBuDPN and rubrene is used as an assist dopant. Concerning the light-emitting layer, the letter % represents % by weight.

In Examples 1 to 9 shown in Table 1, each layer is formed by a vacuum deposition method.

### COMPARATIVE EXAMPLES 1 and 2

As shown in Table 2, Comparative Examples 1 uses diphenylpentacene as a luminescent dopant; Comparative Example 2 uses Compound 4 as a luminescent dopant and tBuDPN as an assist dopant.

Diphenylpentacene has the following structural formula:

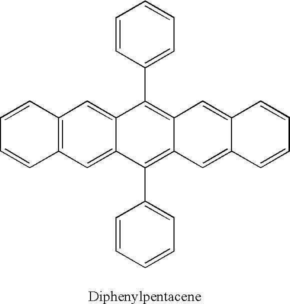

Compound 4 has the following structural formula: 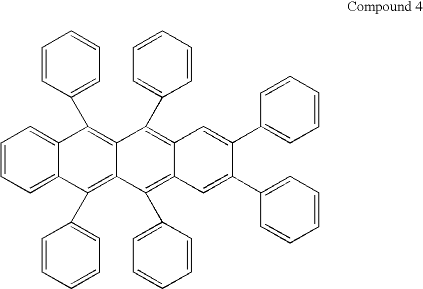
Evaluation of Light-Emitting Properties

The organic EL devices of Examples 1 to 9 and Comparative Examples 1 and 2 as shown in Tables 1 and 2 were each evaluated for light-emitting properties. The results of the evaluation are also shown in Tables 1 and 2. The lifetime represents a half life period for which 1500 cd/m² is reduced to 750 cd/m².

TABLE 1

| | Hole- | Hole- | | Electron- | | | Efficiency | Voltage | Color of | Lifetime |
|---|---|---|---|---|---|---|---|---|---|---|
| | Injecting | Transporting | | Transporting | | | at 20 | at 20 | Emitted | (Half Life) |
| Ex. | Layer | Layer | Light-Emitting Layer | Layer | Cathode | | mA/cm² | mA/cm² | Light | at 1500 cd/m² |
| 1 | CFx | NPB(150) | NPB + 3% Compound 1(30) | Alq(30) | LiF(1) | Al(250) | 4.1 cd/A | 7.0 V | Red-Orange | 1500 hr |
| 2 | CFx | NPB(150) | NPB + 3% Compound 2(30) | Alq(30) | LiF(1) | Al(250) | 4.2 cd/A | 7.3 V | Red-Orange | 1600 hr |
| 3 | CFx | NPB(150) | NPB + 3% Compound 3(30) | Alq(30) | LiF(1) | Al(250) | 3.8 cd/A | 7.4 V | Red-Orange | 1400 hr |
| 4 | CFx | NPB(150) | NPB + 3% Compound 1 + 20% | Alq(30) | LiF(1) | Al(250) | 5.8 cd/A | 6.8 V | Red-Orange | 1800 hr |
| | | | tBuDPN(30) | | | | | | | |
| 5 | CFx | NPB(150) | NPB + 3% Compound 2 + 20% | Alq(30) | LiF(1) | Al(250) | 5.7 cd/A | 6.7 V | Red-Orange | 1900 hr |
| | | | tBuDPN(30) | | | | | | | |
| 6 | CFx | NPB(150) | NPB + 3% Compound 3 + 20% | Alq(30) | LiF(1) | Al(250) | 4.6 cd/A | 6.8 V | Red-Orange | 1700 hr |
| | | | tBuDPN(30) | | | | | | | |
| 7 | CFx | NPB(150) | NPB + 3% Compound 1 + 20% | Alq(30) | LiF(1) | Al(250) | 6.3 cd/A | 6.5 V | Red-Orange | 2100 hr |
| | | | Rubrene(30) | | | | | | | |
| 8 | CFx | NPB(150) | NPB + 3% Compound 2 + 20% | Alq(30) | LiF(1) | Al(250) | 6.4 cd/A | 6.5 V | Red-Orange | 2250 hr |
| | | | Rubrene(30) | | | | | | | |
| 9 | CFx | NPB(150) | NPB + 3% Compound 3 + 20% | Alq(30) | LiF(1) | Al(250) | 5.0 cd/A | 6.6 V | Red-Orange | 1900 hr |
| | | | Rubrene(30) | | | | | | | |

TABLE 2

| | Hole- | Hole- | | Electron- | | | Efficiency | Voltage | Color of | Lifetime |
|---|---|---|---|---|---|---|---|---|---|---|
| Comp. | Injecting | Transporting | | Transporting | | | at 20 | at 20 | Emitted | (Half Life) |
| Ex. | Layer | Layer | Light-Emitting Layer | Layer | Cathode | | mA/cm² | mA/cm² | Light | at 1500 cd/m² |
| 1 | CFx | NPB(150) | NPB + 3% | Alq(30) | LiF(1) | Al(250) | 0.9 cd/A | 8.0 V | Red-Orange | 550 hr |
| | | | Diphenylpentacene(30) | | | | | | | |
| 2 | CFx | NPB(150) | NPB + 3% Compound 4 + | Alq(30) | LiF(1) | Al(250) | 3.6 cd/A | 7.5 V | Red-Orange | 1000 hr |
| | | | 20% tBuDPN(30) | | | | | | | |

The results as shown in Table 1 indicate that the light-emitting layer containing the luminescent material according to the present invention emits red-orange light. A comparison between Tables 1 and 2 indicates that the luminescent material according to the present invention can form an organic EL device having high luminous efficiency and good life properties.

1. A luminescent material represented by General Formula (1): 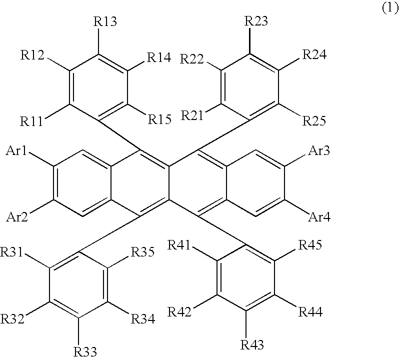
wherein R11 to R15, R21 to R25, R31 to R35, and R41 to R45 are identical to or different from each other and are each hydrogen, an alkyl group represented by —CₙH₂ₙ₊₁ wherein n is an integer of 1 to 10, an alkoxy group represented by —OCₙH₂ₙ₊₁ wherein n is an integer of 1 to 10, a dialkylamino group represented by —N(CₙH₂ₙ₊₁)₂ wherein n is an integer of 1 to 10, fluorine, chlorine, bromine, iodine, a cyano group, or a heterocyclic group represented by Formula (2): 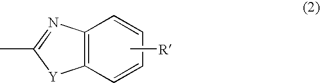
wherein Y is oxygen (O) or sulfur (S); and R′ is hydrogen, an alkyl group represented by —CₙH₂ₙ₊₁ wherein n is an integer of 1 to 10, an alkoxy group represented by —OCₙH₂ₙ₊₁ wherein n is an integer of 1 to 10, a dialkylamino group represented by —N(CₙH₂ₙ₊₁)₂ wherein n is an integer of 1 to 10, fluorine, chlorine, bromine, iodine, a cyano group, or a phenyl group; at least one of R11 to R15, R21 to R25, R31 to R35, and R41 to R45 is the heterocyclic group; and Ar1 to Ar4 are identical to or different from each other and are each an aromatic substituent selected from the following group: 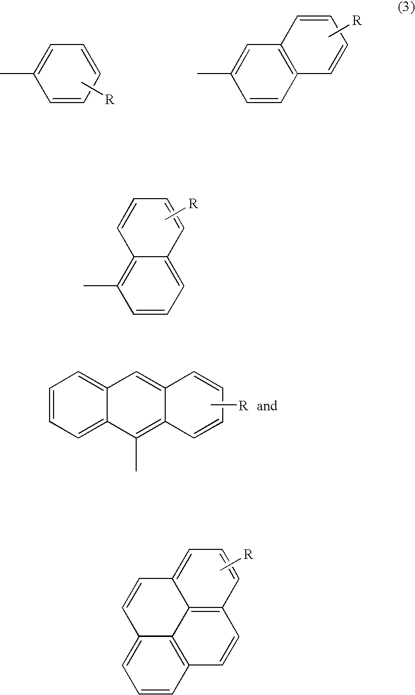
wherein R is hydrogen, an alkyl group represented by —CₙH₂ₙ₊₁ wherein n is an integer of 1 to 10, an alkoxy group represented by —OCₙH₂ₙ₊₁ wherein n is an integer of 1 to 10, a dialkylamino group represented by —N(CₙH₂ₙ₊₁)₂ wherein n is an integer of 1 to 10, fluorine, chlorine, bromine, iodine, a cyano group, or a phenyl group.

2. A luminescent material represented by Formula (4):

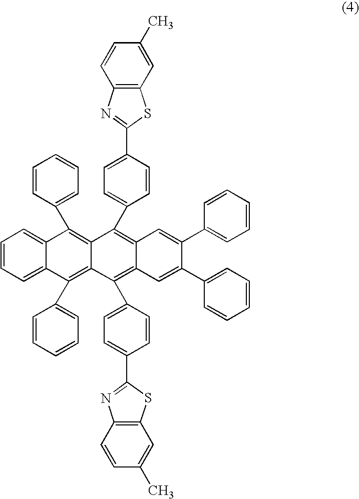

3. A luminescent material represented by Formula (5):

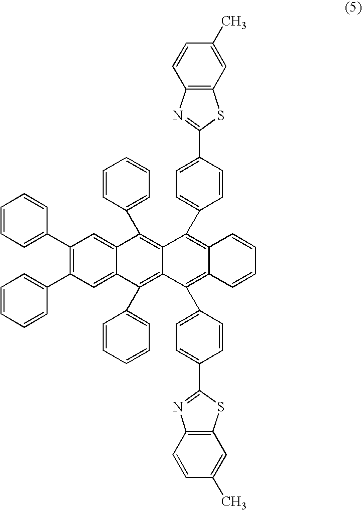

4. A luminescent material represented by Formula (6):

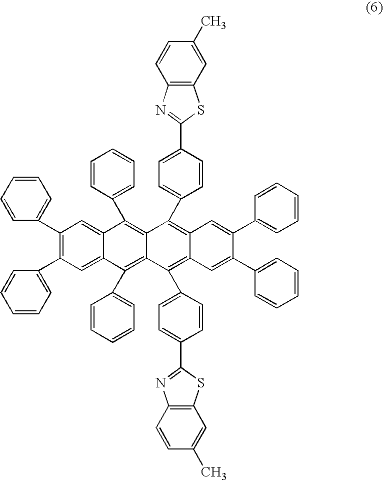

5. An organic electroluminescent device, comprising: a pair of electrodes; and a light-emitting layer sandwiched between the pair of electrodes, wherein the light-emitting layer is a red-orange light-emitting layer and contains the luminescent material of claim 1.

6. The organic electroluminescent device of claim 5, further comprising a green light-emitting layer and a blue light-emitting layer in addition to the red-orange light-emitting layer, whereby the device is capable of full-color display.

7. An organic electroluminescent device, comprising: a pair of electrodes; and a light-emitting layer sandwiched between the pair of electrodes, wherein the light-emitting layer is a red-orange light-emitting layer and contains the luminescent material of claim 2.

8. The organic electroluminescent device of claim 7, further comprising a green light-emitting layer and a blue light-emitting layer in addition to the red-orange light-emitting layer, whereby the device is capable of full-color display.

9. An organic electroluminescent device, comprising: a pair of electrodes; and a light-emitting layer sandwiched between the pair of electrodes, wherein the light-emitting layer is a red-orange light-emitting layer and contains the luminescent material of claim 3.

10. The organic electroluminescent device of claim 9, further comprising a green light-emitting layer and a blue light-emitting layer in addition to the red-orange light-emitting layer, whereby the device is capable of full-color display.

11. An organic electroluminescent device, comprising: a pair of electrodes; and a light-emitting layer sandwiched between the pair of electrodes, wherein the light-emitting layer is a red-orange light-emitting layer and contains the luminescent material of claim 4.

12. The organic electroluminescent device of claim 11, further comprising a green light-emitting layer and a blue light-emitting layer in addition to the red-orange light-emitting layer, whereby the device is capable of full-color display.
